(19) United States
(12) Patent Application Publication
(10) Pub. No.: US 2025/0215458 A1
(43) Pub. Date: Jul. 3, 2025

(54) Production of reverse transcribed DNA (RT-DNA) using a retron reverse transcriptase from exogenous RNA

(71) Applicants: THE J. DAVID GLADSTONE INSTITUTES, A TESTAMENTARY TRUST ESTABLISHED UNDER THE WILL OF J. DAVID, San Francisco, CA (US); THE REGENTS OF THE UNIVERSITY OF CALIFORNIA, OAKLAND, CA (US)

(72) Inventors: Seth Shipman, San Francisco, CA (US); Sierra Lear, Oakland, CA (US)

(21) Appl. No.: 18/850,964

(22) PCT Filed: Mar. 24, 2023
(86) PCT No.: PCT/US2023/016317

§ 371 Date: Sep. 25, 2024

Related U.S. Application Data
(60) Provisional application No. 63/323,537, filed on Mar. 25, 2022.

(51) Int. Cl. C12N 15/90; C12N 9/12; C12N 9/22; C12N 15/10; C12N 15/11

(52) U.S. Cl. CPC C12N 15/907; C12N 9/1276; C12N 9/22; C12N 15/1096; C12N 15/111; C12N 2310/20; C12N 2310/531; C12Y 207/07049

(57) ABSTRACT

Described herein are methods and compositions for transfecting retron RNAs. In vitro transcription can be used to synthesize the retron RNA. The methods and compositions facilitate precise engineering of retron RNAs and can readily be adapted to generate an abundance of different donor DNAs and guide RNAs within cells.

### PRIORITY APPLICATION

This application claims the benefit of priority to U.S. Provisional Patent Application Ser. No. 63/323,537, filed Mar. 25, 2022, the content of which is incorporated herein by reference in its entirety.

### INCORPORATION BY REFERENCE OF SEQUENCE LISTING

A Sequence Listing is provided herewith as an xml file, “2319452.xml” created on Mar. 22, 2023, and having a size of 17,543 bytes. The content of the xml file is incorporated by reference herein in its entirety.

### BACKGROUND

Exogenous DNA can be introduced into cells as a template to edit the cell's genome. However, the amounts of exogenous DNA that can be introduced into cells are limited and not all cells will be transformed by the exogenous DNA. Moreover, it is often unclear which type of exogenous DNA can optimally edit a genomic site and which target site works best for editing.

### SUMMARY

The methods and compositions facilitate precise engineering of retron RNAs and can readily be adapted to provide an abundance of different donor DNAs and guide RNAs in vivo for efficient genomic engineering of the cells.

One embodiment provides a method to produce one or more cells containing reverse transcribed DNA (RT-DNA) copies of at least one retron non-coding RNA (ncRNA) comprising contacting one or more cells with an RNA transcript coding for at least one retron non-coding RNA (ncRNA), wherein the one or more cells express a reverse transcriptase (RT). In one embodiment, the one or more cells stably or transiently expresses RT. In another embodiment, the RT is expressed from an expression plasmid introduced into the one or more cells. In one embodiment, the RT is expressed from an RNA transcript coding for RT, wherein the RNA transcript coding for RT is synthesized in vitro prior to introducing said RNA transcript into the one or more cells. One embodiment further provides contacting the cells with an RNA transcript coding for an mRNA-guided nuclease and/or an RNA transcript coding for guide RNA. In one embodiment, the RNA transcript coding for the at least one retron ncRNA and the RNA transcript coding for RT are present on the same RNA transcript. In another embodiment, the RNA transcript coding for the at least one retron ncRNA, the RNA transcript coding for RT and the RNA transcript coding for an mRNA-guided nuclease are present on the same RNA transcript. In one embodiment, the RNA transcript coding for the at least one retron ncRNA, the RNA transcript coding for RT, the RNA transcript coding for an mRNA-guided nuclease and the RNA transcript coding for guide RNA are present on the same RNA transcript. In another embodiment, the RNA transcript coding for the at least one retron ncRNA, the RNA transcript coding for RT, the RNA transcript coding for an m RNA-guided nuclease and the RNA transcript coding for guide RNA are individually present on separate RNA transcripts. In one embodiment, the retron ncRNA is a modified retron ncRNA compared to a wild type retron non-coding RNA. In another embodiment, the modified retron ncRNA comprises a sequence for a donor DNA. In one embodiment, the RT is a retron RT.

One embodiment provides for a method of genetically modifying a cell comprising: introducing an RNA transcript coding for a modified retron non-coding RNA (ncRNA), an mRNA transcript coding for a retron reverse transcriptase, an RNA transcript coding for an mRNA-guided nuclease and an RINA transcript coding for guide RNA into the cell, wherein the modified retron ncRNA transcript comprises a sequence for a donor DNA; wherein the RNA transcript(s) are synthesized in vitro prior to introducing said RNA transcript(s) into cells, wherein the RNA-guided nuclease forms a complex with the guide RNA, wherein said guide RNA directs the complex to a genomic target locus, wherein the RNA-guided nuclease creates a double-stranded break in genomic DNA of the cell at the genomic target locus, and the donor sequence becomes integrated at the genomic target locus of the cell. In one embodiment, the sequence for the donor DNA comprise one or more variant nucleotides compared to the genomic DNA target locus sequence. In some embodiments single or multiple (successive or simultaneous) rounds of genomic editing can be carried out. One embodiment provides that each guide RNA recognizes and can bind to a genomic DNA gRNA binding site within 100 to 1000 nucleotides of a genomic DNA target site to be edited. In one embodiment, the RNA transcript coding for the retron ncRNA and the RNA transcript coding for RT are present on the same RNA transcript. In another embodiment, the RNA transcript coding for the retron ncRNA, the RNA transcript coding for RT and the RNA transcript coding for the mRNA-guided nuclease are present on the same RNA transcript. In one embodiment, the RNA transcript coding for the retron ncRNA, the RNA transcript coding for RT, the RNA transcript coding for the mRNA-guided nuclease and the RNA transcript coding for the guide RNA are present on the same RNA transcript. In one embodiment, the RNA transcript coding for the retron ncRNA, the RNA transcript coding for RT, the RNA transcript coding for the mRNA-guided nuclease and the RNA transcript coding for the guide RNA are individually present on separate RNA transcripts. In another embodiment, the RT is a retron RT.

In one embodiment, the mRNA-guided nuclease is Cas nuclease. In one embodiment, the Cas nuclease is Cas 9.

In one embodiment, the modified ncRNA comprises an elongated stem region. In another embodiment, the modified ncRNA comprises an elongated loop region. In one embodiment, the modified ncRNA comprises a heterologous RNA. In one embodiment, the heterologous RNA comprises at least one guide RNA sequence. In one embodiment, the heterologous RNA comprises at least an RNA sequence for a donor DNA. In one embodiment, the heterologous RNA comprises at least an RNA sequence, which upon reverse transcription comprises a donor DNA. In one embodiment, the donor DNA is homologous to a target chromosomal sequence in the cell.

In another embodiment, the cells comprise eukaryotic cells. In one embodiment, the cells comprise yeast cells, insect cells, avian cells, or a combination thereof. In one embodiment, the cells comprise mammalian cells. In one embodiment, the cells comprise primary cell lines, immortalized cell lines, cancer cells, stem cells or progenitor cells. In one embodiment, the cells comprise immune cells, pancreatic cells, neuronal cells, liver cells, cardiac cells, bone-generating cells, cartilage-generating cells, skin cells, muscle cells, reproductive cells, kidney cells, lymphocytes, immune cells, or a combination thereof.

### DESCRIPTION OF THE FIGURES

FIG. 1A-1B illustrate that little or no retron ncRNA is introduced into cells when nucleofection is used to transfect cells. FIG. 1A is a control transfection showing that no detectable levels of reverse transcribed DNA (RT-DNA) are produced by cells in the absence of ncRNA delivery to the cells. FIG. 1B shows that no detectable levels of reverse transcribed DNA (RT-DNA) are produced by cells when different amounts of retron ncRNA are transfected into cells by nucleofection.

FIG. 2A-2B illustrate that Lipofectamine™-based methods are useful for introducing retron ncRNA into cells. FIG. 2A shows the amounts of reverse transcribed DNA (RT-DNA) generated when various amounts of ncRNA (0 to 0.1 nmol) are transfected into cells using Lipofectamine™-based methods. FIG. 2B shows the amounts of reverse transcribed DNA (RT-DNA) generated when small amounts of ncRNA (0 to 0.01 nmol) are transfected into cells using Lipofectamine™-based methods. As shown, Lipofectamine™ methods of transfecting RNA into cells are efficient and can provide dose-dependent RNA transfection.

FIG. 3A-3G illustrate evaluation of ncRNA/RT-DNA features as well as retron similarities and differences. FIG. 3A shows a schematic of Eco1 and Eco4 ncRNAs, illustrating a difference between them in the loop identified as having positions 1-3. Both have a1/a2 and stem-loop regions that can be modified as described herein (the a1/a2 regions are labeled and the stem-loop regions are shown in blue). FIG. 3B illustrates the relative abundance of RT-DNA from Eco1 variants having modified loop bases at positions 1-3 of the loop shown in FIG. 3A. Deeper red shades indicate more RT-DNA production. As illustrated, use of thymine in the DNA encoding the ncRNA (before expression), or uracil in the ncRNA, at position 2 in the loop indicated in FIG. 3A of the Eco1 retron ncRNA provides improved RT-DNA (donor DNA) production. FIG. 3C illustrates the relative abundance of RT-DNA from Eco4 variants having modified loop bases at the positions indicated in FIG. 3A. Deeper red shades indicate more RT-DNA production. As illustrated, use of thymine in the DNA encoding the ncRNA, or uracil in the ncRNA, at position 2 in the loop indicated in FIG. 3A does not improve RT-DNA (donor DNA) production in the Eco4 ncRNA as much as it does for the Eco1 ncRNA. Eco4 is less vulnerable than Eco1 to sequence alterations in this loop. FIG. 3D graphically illustrates the relative RT-DNA abundance of each Eco1 stem length variant analyzed, where the RT-DNA abundance is shown as a percentage of wild-type abundance (dashed line). As illustrated, the RT-DNA abundance varies depending upon the length of the stem up to about stem length 15. FIG. 3E graphically illustrates the relative RT-DNA abundance of different Eco1 a1/a2 stem length variants as a percentage of wild-type abundance (dashed line). FIG. 3F graphically illustrates the relative RT-DNA abundance of each Eco4 stem length variant as a percentage of wild-type abundance (dashed line). FIG. 3G graphically illustrates the relative RT-DNA abundance of different Eco4 a1/a2 length variants as a percentage of wild-type abundance (dashed line).

FIG. 4A-4B illustrate modified retrons that can be used in genome editing. FIG. 4A shows a schematic of an RT-DNA template for recombineering. The retron ncRNA was modified in the msd region (blue) to include a long loop (green) that contains homology to a bacterial genomic locus but has one or more nucleotide modifications (repair nucleotides; asterisks). Such an ncRNA therefore provide a template for a donor DNA that is made by reverse transcription. FIG. 4B graphically illustrates fold enrichment of reverse transcribed DNA (RT-DNA) when an Eco1-based retron ncRNA has a longer stem (a1/a2) region of 22 nucleotides, compared to just 12 nucleotides. The RT-DNA products were detected using qPCR, with the RT-DNA from each construct shown relative to uninduced. Circles show each of three biological replicates, with black for the wild type a1/a2 length and green for the extended a1/a2. See FIG. 3 for location of the retron a1/a2 region.

FIG. 5. Editing rates using retron ncRNA/gRNAs provided as exogenous RNA. RNAs were in vitro transcribed and electroporated into HEK293T cells expressing Cas9 and a retron RT of the type matching the RNA (retron-Eco1, retron-Eco3, and retron-Eco5, respectively). In the Eco1 condition, three bases in the target region were changed (EMX1). In the Eco3 and Eco5 conditions, unique 10 base barcodes were inserted at the target site (EMX1). Precise editing rate was determined by amplifying the genomic site three days after electroporation and Illumina sequencing the resulting amplicons. Negative controls (orange) were an identical experiment performed in a cell line expressing only Cas9 (no retron RT).

### DETAILED DESCRIPTION

As described herein, retron non-coding RNA (ncRNA) produced in vitro can be delivered as RNA into cells and then reverse transcribed to efficiently produce multiple copies of the reverse transcribed DNA (RT-DNA). Such methods can, for example, solve low genomic editing problems because the retron nc-RNA is delivered efficiently and the RT-DNA made therefrom is produced inside cells in high abundance.

Retron ncRNAs

The present disclosure provides methods for efficiently introducing retron ncRNAs acids, retron ncRNA variants, retron ncRNA mutants, engineered ncRNAs, or combinations thereof into cells. The retron ncRNAs can in some cases also be modified to include useful exogenous or heterologous nucleic acids, thereby allowing production in vivo of substantial amounts of products such as gRNAs, templates for genomic repair, templates for reverse transcriptases, and the like.

Retrons in nature generally include two elements, one that encodes a reverse transcriptase and a second that is single-stranded DNA/RNA hybrid called a multicopy single-stranded DNA (msDNA). Wild type retrons are about 2 kb long. They contain a single operon controlling the synthesis of an RNA transcript carrying three loci, msr, msd, and ret, that are involved in msDNA synthesis. The DNA portion of msDNA is encoded by the msd gene, the RNA portion is encoded by the msr gene, while the product of the ret gene is a reverse transcriptase. The retron msr RNA is a non-coding RNA (ncRNA) produced by retron elements and is the immediate precursor to the synthesis of msDNA.

While msDNA and reverse transcribed DNA (RT-DNA) are related, the term reverse transcribed DNA (RT-DNA) is used herein to refer to any retron-related reverse transcribed DNA, whether modified or not, while the term msDNA refers to wild type, natural, or unmodified retron msDNA.

The ncRNA includes a pre-msr sequence, an msr gene encoding multicopy single-stranded RNA (msRNA). The msd gene encodes a multicopy single-stranded DNA (msDNA), the post-msd sequence, and a ret gene encoding a reverse transcriptase. Synthesis of DNA by the retron-encoded reverse transcriptase provides a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

For example, a wild type retron-Eco1 ncRNA (also called ec86 or retron-Eco1 ncRNA) can have the sequence shown below as SEQ ID NO:1.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

An example of an Eco1 human-codon optimized reverse transcriptase (RT) sequence that can be used is shown below as SEQ ID NO:2.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

An example of an Eco2 human-codon optimized reverse transcriptase (RT) sequence is shown below as SEQ ID NO:3.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTIGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGTTGGA |
| 921 | GGGTATGTTT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

An example of an Eco1 wild-type retron reverse transcriptase sequence is shown below as SEQ ID NO:4.

| | |
|---|---|
| 1 | KSAEYLNTER LRNLGLPVMN NLHDMSKATR ISVETLRLLI |
| 41 | YTADFRYRIY TVEKKGPEKR MRTIYQPSRE LKALQGWVLR |
| 81 | NILDKLSSSP FSIGFEKHQS ILNNATPHIG ANFILNIDLE |
| 121 | DFFPSLTANK VFGVFHSLGY NRLISSVLTK ICCYKNLLPQ |
| 161 | GAPSSPKLAN LICSKLDYRI OGYAGSRGLI YTRYADDLTL |
| 201 | SAQSMKKVVK ARDFLFSIIP SEGLVINSKK TCISGPRSQR |
| 241 | KVTGLVISQE KVGIGREKYK EIRAKIHHIF CGKSSEIEHV |
| 281 | RGWLSFILSV DSKSHRRLIT YISKLEKKYG KNPLNKAKT |

An example of an Eco2 wild-type retron reverse transcriptase sequence is shown below as SEQ ID NO:5.

| | |
|---|---|
| 1 | MTKTSKLDAL RAATSREDLA KILDIKLVFL TNVLYRIGSD |
| 41 | NQYTQFTIPK KGKGVRTISA PTDRLKDIQR RICDLLSDCR |
| 81 | DEIFAIRKIS NNYSFGFERG KSIILNAYKH RGKQIILNID |
| 121 | LKDFFESENF GRVRGYFLSN QDELLNPVVA TTLAKAACYN |
| 161 | GTLPQGSPCS PIISNLICNI MDMRLAKLAK KYGCTYSRYA |
| 201 | DDITISTNKN TFPLEMATVQ PEGVVLGKVL VKEIENSGFE |
| 241 | INDSKTRLTY KTSRQEVTGL TVNRIVNIDR CYYKKTRALA |
| 281 | HALYRTGEYK VPDENGVLVS GGLDKLEGMF GFIDQVDKEN |
| 321 | NIKKKLNKQP DRYVLTNATL HGFKLKLNAR EKAYSKFIYY |
| 361 | KFFHGNTCPT IITEGKTDRI YLKAALHSLE TSYPELFREK |
| 401 | TDSKKKEINL NIFKSNEKTK YFLDLSGGTA DLKKFVERYK |
| 441 | NNYASYYGSV PKQPVIMVLD NDTGPSDLLN FLRNKVKSCP |
| 481 | DDVTEMRKMK YIHVFYNLYI VLTPLSPSGE QTSMEDLEPK |
| 521 | DILDIKIDGK KENKNNDGDS KTEYGKHIFS MRVVRDKKRK |
| 561 | IDFKAFCCIF DAIKDIKEHY KLMLNS |

An example of a sequence for an Eco4 retron reverse transcriptase is shown below as SEQ ID NO:6.

| | |
|---|---|
| 1 | MSIDIETTLQ KAYPDFDVLL KSRPATHYKV YKIPKRTIGY |
| 41 | RIIAQPTPRV KAIQRDIIEI LKQHTHIHDA ATAYVDGKNI |
| 81 | LDNAKIHQSS VYLLKLDLVN FENKITPELL FKALARQKVD |
| 121 | ISDTNKNLLK QFCFWNRTKR KNGALVLSVG APSSPFISNI |
| 161 | VMSSFDEEIS SFCKENKISY SRYADDLTFS TNERDVLGLA |
| 201 | HQKVKTTLIR FFGTRIIINN NKIVYSSKAH NRHVTGVTLT |
| 241 | NNNKLSLGRE RKRYITSLVF KFKEGKLSNV DINHLRGLIG |
| 281 | FAYNIEPAFI ERLEKKYGES TIKSIKKYSE GG |

An example of a sequence for a Sen2 retron reverse transcriptase is shown below as SEQ ID NO:7.

| | |
|---|---|
| 1 | MDILQHISDL LLTKKSEIIS FSLTAPYRYK IYKIAKRNSD |
| 41 | KKRTIAHPSK ELKFIOREIT EYLTDKLPVH ECAFAYKKGS |
| 81 | SIKTNAQVHL HTKYLLKMDF ENFFPSITPR LFFSKLRLAN |
| 121 | IDLTADDKVL LENILFFKSK RNSNLRLSIG APSSPLISNE |
| 161 | VMYFWDIEVQ EICSKIGVNY TRYADDLTFS TNNKDVLEDI |
| 201 | PDMLENVLPK YSLGRIRINH EKTVFSSKGH NRHVTGITLT |
| 241 | NDNKLSIGRE RKRKISAMIH HFINGKLSTD ECNKLVGLLA |
| 281 | FAKNIEPSFY KSMVIKYGSD NIYKLOKQKD K |

Other types of retron types and retron components are described throughout the application and can be used in the methods described herein.

In some cases, the ncRNA has the secondary structures of the retron ncRNA are substantially preserved even when modifications are present in the ncRNAs. In addition, the a1/a2 region should also be preserved. This means that while RNA stem and loop features as well as the a1/a2 regions can be modified (e.g., lengthened), such stem and loop features and such a1/a2 regions should not be entirely deleted or be so destabilized that the integrity of these secondary structures is lost. In other words, the secondary structures of the ncRNA should not be so destabilized that it becomes degraded either during in vitro preparation or in vivo.

Modified (e.g., engineered) ncRNAs can have alterations in different locations relative to the corresponding wild type ncRNAs. However, not every modification provides a stable ncRNA or one that can yield good amounts of reverse transcribed DNA.

One example of a location for modification of retron ncRNA is within a self-complementary region (stem region, which has sequence complementarity to the pre-msr sequence), wherein the length of the self-complementary region can be lengthened relative to the corresponding ncRNA of a native retron. Such modifications should retain the complementarity of the stem structure.

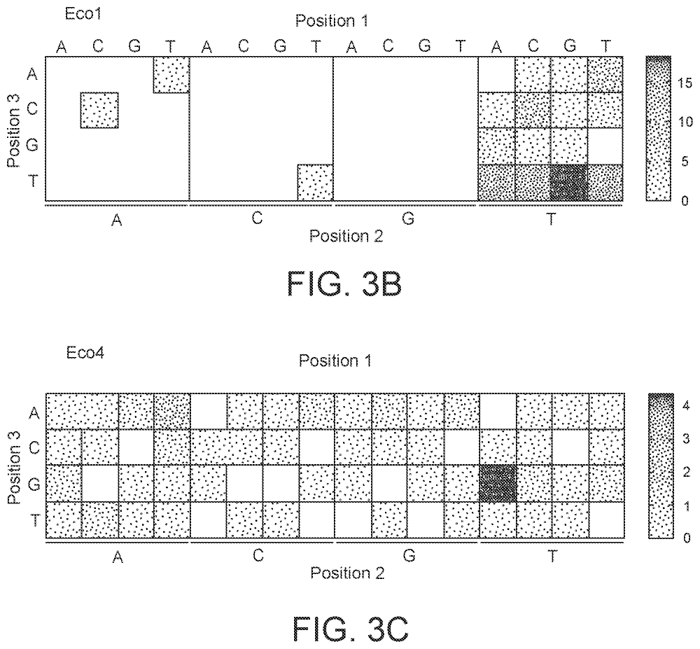

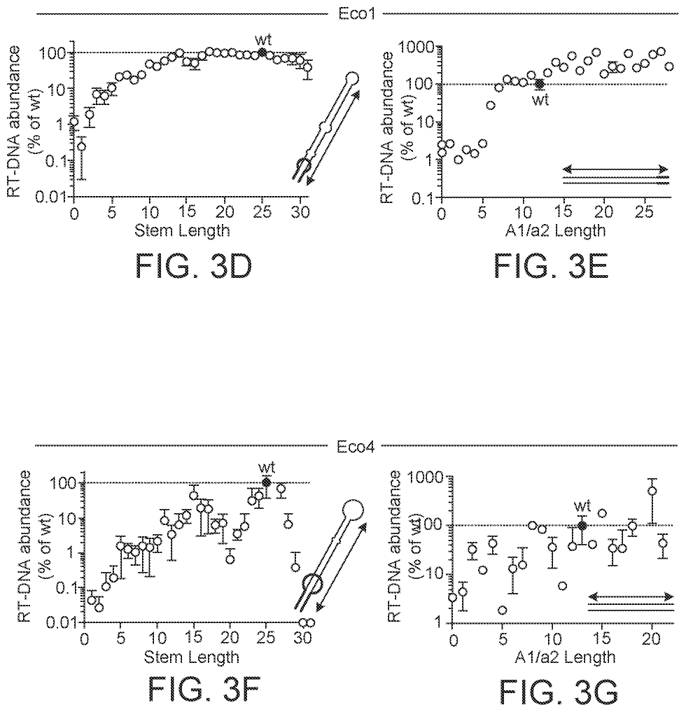

Lengthening the stem results in an engineered retron that can provide enhanced production of RT-DNA (see, e.g., FIG. 3).

In certain embodiments, the complementary stem region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from 1 to 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, 50, or more nucleotides longer. In certain embodiments, the self-complementary region has a length ranging from 1 to 16 nucleotides longer than the wild-type complementary region. The single-stranded DNA generated by the engineered retron ncRNA can be used in various applications.

To create more abundant RT-DNA, for example, the ncRNA SEQ ID NO:8 sequence shown below, with the native self-complementary 3′ and 5′ ends highlighted in bold (at positions 1-12 and 158-169), can be extended at positions 1 and 169 to extend the self-complementary region.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

For example, as shown below for the following engineered “ncRNA extended” (SEQ ID NO:9), where the additional nucleotides that extend the self-complementary region are shown in italics with underlining.

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACTGTCT GTTTTCCTTG TTGGAACGGA |
| 121 | GAGCATCGCC TGATGCTCTC CGAGCCAACC AGGAAACCCG |
| 161 | TTTTTTCTGA CGTAAGGGTG CGCATACGGA ATCTTATCA |

In some cases, the additional nucleotides can be added to any position in the self-complementary region, for example, anywhere within positions 1-12 and 158-169 of the SEQ ID NO:8 or SEQ ID NO:9 sequence.

In certain embodiments, sequences of the ncRNA, msr gene, msd gene, and ret gene used in the engineered retron may be derived from any bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica; Vibrio cholerae retrons (e.g., Vc81, Vc95, Vc137); Vibrio parahaemolyticus (e.g., Vc96); and Nannocystis exedens retrons (e.g., Ne144). Retron ncRNA, msr gene, msd gene, and ret gene nucleic acid sequences as well as retron reverse transcriptase protein sequences may be derived from any source. Representative retron sequences, including ncRNA, msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties.

The retron ncRNAs can be modified to enhance production of retron reverse transcribed DNA in a host cell or to provide host cells with genomic editing components or other useful proteins and/or nucleic acids. Any of the foregoing retron sequences (or variants thereof) can include variant or mutant nucleotides, added nucleotides, or fewer nucleotides.

For example, a parental ncRNA can be modified by addition of nucleotides to a stem or loop as described herein. Before modification the parental ncRNA can have at least about 80-100% sequence identity to any region of the retrons described herein, including any percent identity within this range, such as 81, 82, 83, 84, 85, 86, 87, 88, 89, 90, 91, 92, 93, 94, 95, 96, 97, 98, or 99% sequence identity to any region of the retron sequences described herein (including those defined by accession number). Such parental retrons can be used to construct an engineered retron or vector system comprising an engineered retron, as described herein.

The variant ncRNAs can include exogenous or heterologous nucleotides or nucleic acid segments. For example, the exogenous or heterologous nucleotide or nucleic acid segments can add at least 1, at least 2, at least 4, at least 6, at least 8, at least 10, at least 12, at least 14, at least 16, at least 18, at least 20, at least 30, at least 40, at least 50, at least 60, at least 70, at least 80, at least 90, at least 100, at least 125, at least 150, at least 175, or at least 200 nucleotides to parental retron nucleic acids, to thereby generate variant retron nucleic acids.

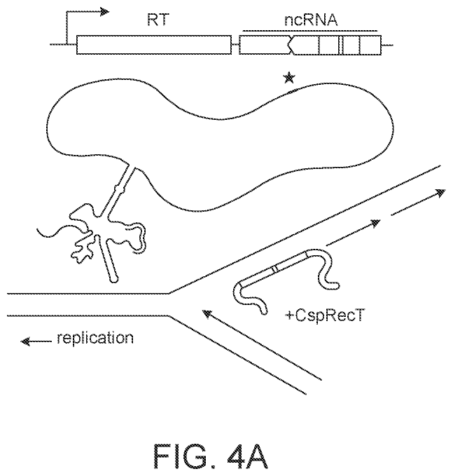

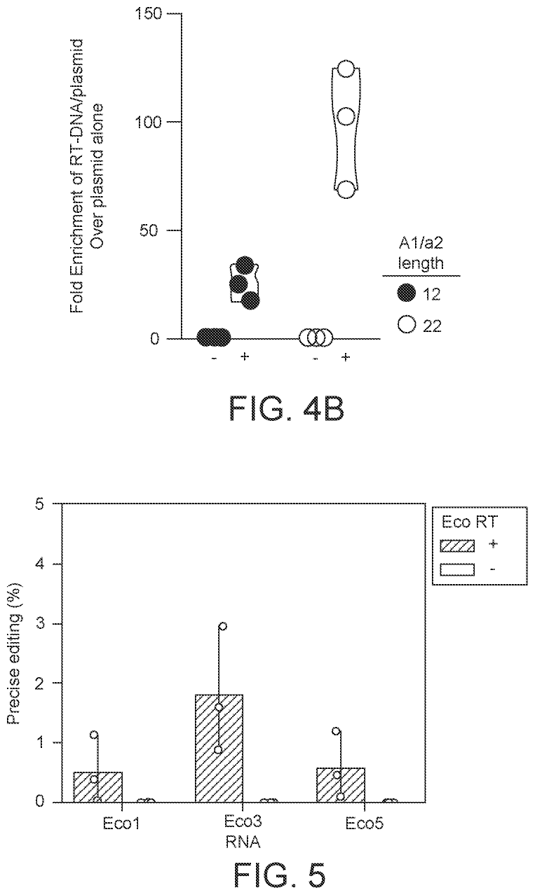

One example of a locus for insertion of exogenous or heterologous nucleotide or nucleic acid segments into retron nucleic acids is a loop portion of a stem-loop (see, e.g., FIG. 4A).

As described above, the retron nucleic acids can be modified with respect to the native retron to include one or more heterologous sequences of interest, including a donor polynucleotide suitable for use in gene editing, e.g., by homology directed repair (HDR) or recombination-mediated genetic engineering (recombineering), a barcode, a guide RNA (e.g., with the tracrRNA), as discussed further below. Such heterologous sequences may be inserted, for example, into the ncRNA coding region in the expression cassette. Upon transcription, the ncRNA will contain the guide RNA, as well as the RNA segment encoding the donor DNA. The ncRNA can be partially reverse transcribed to generate the donor RNA.

In some cases, the donor DNA sequence of interest can be inserted into the loop of the msd stem loop of the retron or a loop of the ncRNA.

In some cases, engineered retron nucleic acids can include unique barcodes to facilitate multiplexing. Barcodes may comprise one or more nucleotide sequences that are used to identify a nucleic acid or cell with which the barcode is associated. Such barcodes may be inserted for example, into the loop region of the msd-encoded DNA. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. A barcode may be used to identify the presence of a particular genetically modified site within a host cell. The use of barcodes allows retron nucleic acids from different cells to be pooled in a single reaction mixture for sequencing while still being able to trace a particular retron, ncRNA, donor DNA, reverse transcriptase, or cas nuclease back to the colony from which it originated.

Retron ncRNA Expression

Provided herein, one or more of RNA transcripts that code for ncRNAs, donor DNAs, barcodes, guide RNAs, reverse transcriptases, and/or cas nucleases can be synthesized in vitro (e.g., in vitro transcription or chemical synthesis) and then the RNA transcripts can be introduced directly to cells (rather than be expressed from expression plasmids introduced into cells. ncRNAs, donor DNAs, guide RNAs, reverse transcriptases, and/or cas nucleases can be present on one more RNA transcripts. If more than one of ncRNAs, donor DNAs, guide RNAs, reverse transcriptases, barcodes, and/or cas nucleases is present on a transcript, 2A, fusion, IRES can be used to add in translation/protein expression.

One or more expression cassettes/plasmids/vectors may also be used. Expression cassettes or expression vectors can used to provide abundant ncRNA by in vitro transcription. Such expression cassettes or expression vectors can include a promoter operably linked to a nucleic acid segment encoding any of the ncRNAs described herein. The segment encoding the ncRNA can include a sequence for a modified ncRNA as described herein (e.g., a modified ncRNA encoding one or more donor DNAs, guide RNAs, or combinations thereof). The constructs can also include a segment encoding one or more proteins. For example, proteins can be expressed from the constructs that are useful as tools to manipulate or process the RT-DNA generated in the cell. Examples of proteins that can be expressed from the expression constructs include reverse transcriptases, cas nucleases, polymerases, or combinations thereof.

Barcodes can be included in the modified ncRNA so that the RT-DNA generated therefrom is incorporated into nucleic acids (e.g., genomic sites) within host cells. For example, the productions and fate of a particular a barcode that is inserted into the genome and can be recovered by sequencing. In this way, many variables can be identified and evaluated in the same population of cells to assess relative integration frequency.

The modified retron constructs can have a non-native configurations with non-native spacing between the ncRNA coding region and the reverse transcriptase (ret) coding region. For example, it can be useful to separate the expression cassettes that include the ncRNA coding region and the reverse transcriptase (ret) coding region. Hence, the ncRNA and the reverse transcriptase may be separated in a trans arrangement rather than provided in the natural cis arrangement. In some embodiments, the ret gene is provided in a trans arrangement that eliminates a cryptic stop signal for the reverse transcriptase, which allows the generation of longer single stranded DNAs from the engineered retron construct.

Amplification of retron/ncRNA encoding nucleic acids may be performed, for example, before they are used as a template for in vitro transcription. In some cases, RT-DNA or genomic DNA isolated from cells after transfection of the ncRNA can amplified. Any method for amplifying the retron nucleic acids may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the retron constructs comprise common 5′ and 3′ priming sites to allow amplification of retron sequences in parallel with a set of universal primers. In another embodiment, a set of selective primers is used to selectively amplify a subset of retron sequences from a pooled mixture.

Abundant amounts of ncRNAs for use in the methods described herein can be provided by in vitro transcription. In general, in vitro transcription can be performed using the following components: DNA template having a promoter operably linked to a nucleic acid encoding the ncRNA, ribonucleotide triphosphates, a buffer system that includes DTT and magnesium ions, and an appropriate RNA polymerase. In some cases, the in vitro transcription mixture can include RNase inhibitors, capping enzymes, mRNA cap 2′-O-methyltransferases, pyrophosphatases, or combinations thereof.

The RNA polymerase can be a viral, mammalian, prokaryotic or phage RNA polymerase. In some cases, the RNA polymerase is a bacteriophage RNA polymerase. For example, the RNA polymerase can be a T7, T3, or SP6 RNA polymerase.

Mammalian RNA polymerases may in some cases be used such as RNA polymerase I (in wild type cells it synthesizes ribosomal RNA), RNA polymerase II (in wild type cells it synthesizes most mRNAs, microRNAs, and small RNAs), or RNA polymerase III (in wild type cells it synthesizes tRNAs, 5S rRNA, and other small RNAs).

The DNA template encoding the ncRNA can be semi-pure or pure, meaning that contaminants such as salts, alcohols, phenol, and the like that may inhibit transcription should be removed. The DNA template should also be a linear DNA. In some cases, the DNA template is optimally free of cryptic phage RNA polymerase termination sites.

Mutant, modified, variant, or wild type retron ncRNAs can individually or collectively be transcribed in vitro from an expression cassette or expression vector. As described herein, heterologous sequences encoding desired products of interest (e.g., guide RNAs, donor polynucleotide for gene editing, barcodes, or combinations thereof) may be inserted in one or segments of the region encoding the ncRNA.

The ncRNA preparations can be produced in vitro and transfected into cells expressing reverse transcriptases, cas nucleases, or other proteins. In some embodiments, the reverse transcriptases, cas nucleases, or other proteins are expressed from expression cassettes having a promoter operably linked to the segment encoding the reverse transcriptase, cas nuclease or other protein. In some cases, a segment encoding the reverse transcriptase may be incorporated into an expression cassette or expression vector that is distinct from an expression cassette or expression vector that includes a promoter operably linked to a segment encoding a cas nuclease or other protein. This allows independent (e.g., separate induction of) expression of the reverse transcriptases and the cas nucleases or other proteins.

The wild type or modified retron ncRNA, reverse transcriptase, cas nuclease or other protein sequences can also be under transcriptional control of a promoter.

A “promoter” refers to a DNA sequence recognized by the synthetic machinery of the cell, or introduced synthetic machinery, required to initiate the specific transcription of a gene. The term promoter used herein refers to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Expression cassette and expression vectors can include a promoter “operably linked” to a nucleic acid segment encoding the ncRNA, a reverse transcriptase, or a cas nuclease. The phrase “operably linked” or “under transcriptional control” as used herein means that the promoter is in the correct location and orientation in relation to a polynucleotide to control the initiation of transcription by RNA polymerase and expression of the ncRNA and/or the reverse transcriptase.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. Such an IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997 22: 150-161. A multitude of IRES sequences are available and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acad. Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363(Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a reverse transcriptase or a cas nuclease (e.g., Cas9) from an expression cassette.

Alternatively, a polynucleotide encoding a viral 2A-self cleaving peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLoS One 6(4):e18556, Trichas et al. (2008) BMC Biol. 6:40, Provost et al. (2007) Genesis 45(10):625-629, Furler et al. (2001) Gene Ther. 8(11):864-873; herein incorporated by reference in their entireties.

In certain embodiments, expression constructs can be within a plasmid suitable for transforming a bacterial, yeast or mammalian host cell. Numerous bacterial expression vectors are available. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31. Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression constructs can be within plasmids suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct can be within a virus or engineered construct derived from a viral genome. A number of viral based systems have been developed to maintain an expression system and express encoded products within mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes (e.g., reverse transcriptases and/or cas nucleases) into mammalian cells.

For example, retroviruses provide convenient expression systems. Selected sequences (e.g., reverse transcriptases and/or cas nucleases) can be inserted into a vector and packaged in retroviral particles. The recombinant virus can then be isolated and delivered to host cells, or cells of a selected subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids such as reverse transcriptases and/or cas nucleases is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest (e.g., reverse transcriptases and/or Cas nucleases) include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there can be modified retron nucleic acids whose expression is under the control of the T7 promoter. Thus, some of the T7 RNA polymerase generated from translation of the amplification template RNA will lead to transcription of the desired retron ncRNAs and/or retron reverse transcriptases. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King, The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant host cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Any cells that can express reverse transcriptases and/or Cas nucleases, for example as described above, can be transfected with the in vitro transcribed ncRNAs. For example, a bacteriophage T7 RNA polymerase can used to transcribe the ncRNA. This polymerase displays exquisite specificity in that it only transcribes templates bearing T7 promoters. The method provides for high level production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126. Thus, a T7 RNA polymerase promoter can be operably connected to a nucleic acid segment encoding an ncRNA. For example, a cDNA encoding the ncRNA can be under the control of the T7 promoter. The T7 RNA polymerase can be introduced as a protein to the in vitro transcription mixtures as described by the methods herein. For a further discussion of T7 systems and their uses, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The methods and compositions therefore allow optimal production and design of improved gene editing and recombinant modification methods. For example, the methods described herein can provide improved/optimal ncRNA chassis, gRNA sequences, gRNA designs, reverse transcriptases, CRISPR nucleases, and combinations thereof.

In order to effect expression of engineered retron constructs, the expression construct or synthesized RNA transcripts(s) can be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Delivery of retron nucleic acids to a cell can generally be accomplished with or without vectors. The retrons, retron nucleic acids, or vectors containing them may be introduced into any type of cell, including any cell from a prokaryotic, eukaryotic, or archaeon organism, including bacteria, archaea, fungi, protists, plants (e.g., monocotyledonous and dicotyledonous plants), and animals (e.g., vertebrates and invertebrates). Examples of animal cells that may be transfected with an engineered retron include, without limitation, cells from vertebrates such as fish, birds, mammals (e.g., human and non-human primates, farm animals, pets, and laboratory animals), reptiles, and amphibians. Examples of plant cells that may be transfected with an engineered retron include, without limitation, cells from crops including cereals such as wheat, oats, and rice, legumes such as soybeans and peas, corn, grasses such as alfalfa, and cotton. The engineered retrons can be introduced into a single cell or a population of cells of interest. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons. The subject methods are also applicable to cellular fragments, cell components, or organelles (e.g., mitochondria in animal and plant cells, plastids (e.g., chloroplasts) in plant cells and algae). Cells may be cultured or expanded after transfection with the engineered retron constructs.

A variety of methods for introducing nucleic acids into a host cell are available. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

Once the expression construct has been delivered into the cell the vector or cassette comprising the retron nucleic acids may be positioned and expressed at different sites. In certain embodiments, the vector or cassette comprising the retron nucleic acids may be stably integrated into the genome of the cell. This integration may be in the cognate location and orientation, or it may be integrated in a random, non-specific location (gene augmentation). In yet further embodiments, the vector or cassette comprising the retron nucleic acids may be stably maintained in the cell as a separate, episomal segment of DNA. Such nucleic acid segments or “episomes” encode sequences sufficient to permit maintenance and replication independent of or in synchronization with the host cell cycle. How the vector or cassette comprising the retron nucleic acids are delivered to a cell and where in the cell the nucleic acid remains is dependent on the type of expression construct employed.

In yet another embodiment, the expression construct may simply consist of naked recombinant DNA or plasmids comprising the retron nucleic acids (e.g., expression cassettes). Transfer of the constructs may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding retron nucleic acids of interest may also be transferred in a similar manner in vivo and express retron products.

In other cases, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinin virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising retron nucleic acids may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

Cell transfection can be carried out by any method available to an art worker, including commercials materials and methods, such as with cationic lipids (lipofection or lipid-mediated/liposome transfection), lipid nanoparticles, non-liposomal methods (e.g., FuGENE™), nucleofection and electroporation. For cationic lipids, this technique uses a positively charged (cationic) lipid/liposomes which are amphiphilic molecules and interact electrostatically with negatively charged (anionic) phosphate residues of DNA/RNA and cell membranes. As described herein exogenous retron RNA is an excellent source of nucleic acids for transfection of cells, particularly when a cationic-lipid transfection reagent is used, such as DOSPA (2,3-dioleoyloxy-N-[2(sperminecarboxamido)ethyl]-N,N-dimethyl-1-propaniminium trifluoroacetate), DOPE (1,2-dioleoyl-sn-glycerophospho-ethanolamine), DOTMA (N-[1-(2,3-dioleyloxy)propyl]-N,N,N-trimethylammonium chloride) or a combination thereof. A commercial example is Lipofectamine™. Lipofectamine™ includes DOSPA (2,3-dioleoyloxy-N-[2(sperminecarboxamido)ethyl]-N,N-dimethyl-1-propaniminium trifluoroacetate) and DOPE (1,2-dioleoyl-sn-glycerophospho-ethanolamine). In some cases, the cationic-lipid transfection reagent can have more DOSPA than DOPE or more DOPE than DOSPA. For example, the DOSPA can be present at twice or three times the amount of DOPE, such as in a ratio of DOPE:DOSPA of 1:1, 1:1.25, 1:1.5, 1:1.75, 1:2, 1:2.25, 1:2.5, 1:2.75, 1:3 or DOSPA:DOPE of 1:1, 1:1.25, 1:1.5, 1:1.75, 1:2, 1:2.25, 1:2.5, 1:2.75, 1:3. The cationic-lipid transfection reagent can also be used with a lipid which helps to coat negatively charged RNA, conferring a positive charge to the RNA-lipid particles or droplets. For example, such a lipid can be used before, simultaneously with, or after the RNA is contacted with the DOSPA/DOPE. Typically, a lipid is used simultaneously, or in combination with, the DOSPA/DOPE. In some cases, the cationic-lipid transfection reagent can be a Lipofectamine™ 2000 or Lipofectamine™ 3000 system (e.g., from Invitrogen or Thermo Fisher).

The methods can therefore include contacting a cationic-lipid transfection reagent with RNA. In some cases, the RNA is in aqueous solution, for example, sterile water or cell culture medium. The amount of RNA contacted by the cationic-lipid transfection reagent for transfection into cells can vary. For example, about 1×10⁻⁷ to about 1 nmol RNA, or about 1×10⁻⁶ to about 0.5 nmol RNA, or about 5×10⁻⁵ to about 0.3 nmol RNA can be mixed with, or can be contacted by, the cationic-lipid transfection reagent to form an RNA-cationic lipid (such as Lipofectamine™) mixture.

The RNA-cationic lipid mixture can be contacted with cells plated or in suspension in a cell media. In some cases, the RNA-cationic lipid mixture can be contacted with plated cells. In other cases, the RNA-cationic lipid mixture can be contacted with cells in suspension. The cells may be sufficiently concentrated together to reduce the volume of RNA and cationic-lipid transfection reagent needed to contact the majority of cells. However, the cells may not be so crowded to inhibit recovery and/or growth of the cells after contact with the RNA-cationic lipid mixture. For example, in some cases, less than confluent plated cells can be contacted with the RNA-cationic lipid. In some cases, plated cells can be about 50% to about 95% confluent, or about 60% to about 95% confluent, or about 70% to about 95% confluent, or about 50% to about 90% confluent, or about 50% to about 85% confluent.

The cells can be incubated with the RNA-cationic lipid mixture for about 6 hours to about 96 hours, or about 10 hours to about 72 hours, or about 16 hours to about 48 hours.

The cells can be eukaryotic cells or prokaryotic cells. However, the cells generally express at least one type of reverse transcriptase. The reverse transcriptase can be a retron reverse transcriptase (e.g., from Eco1, Eco2, or any of the retrons described herein), a viral reverse transcriptase (e.g., from human immunodeficiency virus (HIV) or avian myeloblastosis virus (AMV)), a bacterial reverse transcriptase (e.g., from myxobacteria or Escherichia coli), or any eukaryotic reverse transcriptase.

Examples of cell types that can be used in the methods described herein include primary cell lines, immortalized cell lines, cancer cells, stem cells, progenitor cells, neuronal cells, pancreatic cells, liver cells, cardiac cells, bone-generating cells, cartilage-generating cells, skin cells, muscle cells, reproductive cells, kidney cells, lymphocytes, immune cells, or a combination thereof.

Cells treated with the RNA-cationic lipid mixture can be evaluated for production of reverse transcribed DNA, or other modifications that may be provided by a modified ncRNA or a reverse transcribed DNA (RT-DNA) copy thereof. For example, the amount of RT-DNA in cells treated with the RNA-cationic lipid mixture can be evaluated or measured by quantitative polymerase chain reaction (qPCR), by electrophoretic separation, by Southern blotting, by ultraviolet spectrometry, by labeled signaling reagents (e.g., fluorescent probes), by diphenylamine, by visible spectrometry, or combinations thereof.

The RNA used for transfection can be any RNA. However, in some cases the RNA is retron ncRNA. Use of ncRNAs is convenient for several reasons. For example, ncRNAs have recognition sites for reverse transcriptases. In another example, ncRNAs have secondary structures that improve their stability. Moreover, the regions of ncRNAs that can be modified without reducing the stability or reverse transcription of the ncRNAs have been defined by the inventors. Hence, additional nucleotide sequences can be incorporated into selected regions of ncRNAs without loss of function.

Therefore, as shown herein, a cationic-lipid transfection reagent, such as Lipofectamine™)-related transfection of RNA, particularly retron ncRNAs, into cells is a useful tool.

Although nucleofection has been used for introduction of a variety of nucleic acids into different cell types, the experiments described herein show that use of Lipofectamine™ provides improved transfection of retron ncRNAs compared to nucleofection. Nucleofection is an electroporation-based transfection method which enables transfer of nucleic acids into cells by applying a specific voltage and reagents.

### Libraries

Libraries of modified nucleic acids (modified ncRNAs or modified DNAs encoding ncRNAs that can include or encode modified reverse transcriptases, different guide RNAs, modified cas nucleases, and combinations thereof) can be used in the methods described herein. Thousands of different types of modified ncRNAs or modified retron ncRNAs DNAs can be synthesized and incorporated into expression vectors. Upon transcription, a population of modified ncRNAs can be generated that can be introduced (transfected) into cells for production of a population of RT-DNAs.

For example, a golden-gate-based cloning strategy (Engler et al., PLOS One (Nov. 5, 2008)) can be used to generate large pools of modified retron ncRNAs. Such modified retron ncRNAs can encode various donor DNAs, different reverse transcriptases, various guide RNAs, different cas nucleases, and combinations thereof can be expressed (transcribed) for transcription in vitro, transfection, and production of RT-DNA.

For example, a plasmid having or encoding a parental ncRNA nucleic acid insert can be subjected to directed mutagenesis to generate a population of plasmids with different nucleic acid inserts that encode the different ncRNAs. Expression cassette encoding the different ncRNAs can be cleaved or amplified from the plasmid so that the ncRNAs can be transcribed in vitro to generate a population of different modified retron ncRNAs.

Alternatively, a population of oligonucleotides encoding ncRNAs (e.g., one that encodes a donor DNA and/or a guide RNA) can be subjected to directed mutagenesis to generate a population of variant oligonucleotide ncRNAs. Such oligonucleotide ncRNAs can be inserted into expression vectors or expression cassettes to operably link them to a promoter and other regulatory sequences. The expression cassette can be excised, or the plasmid linearized so that the ncRNAs can be transcribed in vitro to generate a population or library of variant ncRNAs. Such variant ncRNAs can include donor DNAs and/or a guide RNAs to provide genomic editing when the ncRNAs are transfected into host cells. The results that occur in host cells expressing a reverse transcriptase and a cas nuclease can be evaluated using sequencing, RT-DNA quantification, and other methods described herein.

### Genomic Editing

The methods described herein can perform genomic editing by using clustered regularly interspaced short palindromic repeats (CRISPR)/CRISPR-associated (Cas) systems. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties).

A CRISPR guide RNA system can be adapted for use in the methods and compositions described herein. Two RNAs can be used in CRISPR genomic editing systems: a CRISPR RNA (crRNA), which is a 17-20 nucleotide sequence complementary to the target DNA, and a trans-activating crRNA (tracrRNA) that is a binding scaffold for the Cas nuclease. In some cases the two RNAs are fused to make a single guide RNA (sgRNA). The tracrRNA forms a stem loop that is recognized and bound by the cas nuclease. The crRNA typically has shorter sequence than the tracrRNA. The term “guide RNA” as used herein refers to either a single guide RNA (sgRNA) or a crRNA. The CRISPR technique is generally described, for example, by Mali et al. Science 339:823-6 (2013); which is incorporated by reference herein in its entirety.

The guide RNA system used herein is encoded within or adjacent to the ncRNA coding region of the expression cassettes. Hence, upon transcription of the guide RNA, it can target a Cas enzyme to the desired location in the genome, where it can cleave the genomic DNA for generation of a genomic modification. Donor DNA encoded within the retron ncRNA and reverse transcribed within the host cells modifies (e.g., repairs) the genomic target site.

There are several types of CRISPR systems, some of which are summarized in the chart below.

CRISPR System Types Overview

| System | Features | Examples |
|---|---|---|
| Type I | Multiple proteins (5-7 proteins | Staphylococcus epidermidis |
| | typical), crRNA. DNA Cleavage | (Type IA) |
| | is catalyzed by Cas3. | |
| Type II | 3-4 proteins (one protein (Cas9) | Streptococcus pyogenes |
| | has nuclease activity) two RNAs. | CRISPR/Cas9, Francisella |
| | Target DNA cleavage catalyzed by | novicida U112 Cpf1 |
| | Cas9 and RNA components. | |
| Type III | Five or six proteins required for | S. epidermidis (Type IIIA); |
| | cutting, number of required RNAs | P. furiosus; (Type IIIB). |
| | unknown but expected to be one. | |
| | Type IIIB systems | |
| | have the ability to | |
| | target RNA. | |

In some cases, the cas nuclease is a Type II CRISPR endonuclease. The term “Class II CRISPR endonuclease” refers to endonucleases that have similar endonuclease activity as Cas9 and participate in a Class II CRISPR system. The Cas9 nuclease can, for example, be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

An example of a Class II CRISPR system is the type II CRISPR locus from Streptococcus pyogenes SF370, which contains a cluster of four genes Cas9, Cas1, Cas2, and Csn1, as well as a tracrRNA and a characteristic array of repetitive sequences (direct repeats) interspaced by short stretches of non-repetitive sequences (spacers, about 30 bp each). In this system, targeted DNA double-strand break (DSB) may be generated in four sequential steps. First, the pre-crRNA array and tracrRNA, may be transcribed from the expression cassette that encodes the ncRNA and the guide RNA. Second, tracrRNA may hybridize to the direct repeats of pre-CRISPR guide RNA (pre-crRNA), which is then processed into mature crRNAs containing individual spacer sequences. Third, the mature crRNA:tracrRNA complex can direct Cas9 to the DNA target consisting of the protospacer and the corresponding PAM sequence via heteroduplex formation between the spacer region of the crRNA and the protospacer DNA. Finally, Cas9 may mediate cleavage of target DNA upstream of PAM to create a double-stranded break within the protospacer.

A “guide RNA” or “gRNA” as provided herein refers to a ribonucleotide sequence capable of binding a cas nuclease, thereby forming ribonucleoprotein complex. The gRNA includes a nucleotide sequence complementary to a target site (e.g., near or at a genomic site to be edited). In some cases, the guide RNA includes one or more RNA molecules. TracrRNAs can be used to facilitate assembly of a ribonucleoprotein complex that includes the gRNA together with the tracrRNA and a cas nuclease. A complementary nucleotide sequence of the guide RNA can mediate binding of the ribonucleoprotein complex to the target site thereby providing the sequence specificity of the ribonucleoprotein complex. Thus, the guide RNA includes a sequence that is complementary to a target nucleic acid sequence such that the guide RNA binds a target nucleic acid sequence.

In some cases, the complement of the guide RNA includes a sequence having a sequence identity of about 50%, 55%, 60%, 65%, 70%, 75%, 80%, 85%, 90%, 95%, 96%, 97%, 98% or 99% to a target nucleic acid (e.g., a target genomic DNA sequence). In some cases, a target nucleic acid sequence is a nucleic acid sequence expressed by a cell. In some cases, the target nucleic acid sequence is an exogenous nucleic acid sequence. In some cases, the target nucleic acid sequence is an endogenous nucleic acid sequence. In some cases, the target nucleic acid sequence forms part of a cellular gene. In some cases, the target nucleic acid sequence is a genomic DNA site or location. Thus, some cases, the guide RNA is complementary to a cellular gene or fragment thereof. In some cases, the guide RNA includes a sequence having sequence identity of about 50%, 55%, 60%, 65%, 70%, 75%, 80%, 85%, 90%, 95%, 96%, 97%, 98% or 99% to the target nucleic acid sequence. In some cases, the guide RNA includes a sequence that is about 50%, 55%, 60%, 65%, 70%, 75%, 80%, 85%, 90%, 95%, 96%, 97%, 98% or 99% complementary to the sequence of a cellular gene. In some cases, the guide RNA binds a cellular gene target sequence. In some cases, the guide RNA or complement thereof, includes a sequence having a sequence identity of at least about 90%, 95%, or 100% to a target nucleic acid.

In some cases, segment bound by a guide RNA within the target nucleic acid is about or at least about 10, 15, 20, 25, or more nucleotides in length.

The guide RNA is a single-stranded ribonucleic acid, although in some cases it may form some double-stranded regions by folding onto itself. In some cases, the guide RNA is about 10, 20, 30, 40, 50, 60, 70, 80, 90, 100 or more nucleic acid residues in length. In some cases, the guide RNA is from about 10 to about 30 nucleic acid residues in length. In some cases, the guide RNA is about 20 nucleic acid residues in length. For example, the length of the guide RNA can be at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides or residues in length. In some cases, the guide RNA is from 5 to 50, 10 to 50, 15 to 50, 20 to 50, 25 to 50, 30 to 50, 35 to 50, 40 to 50, 45 to 50, 5 to 75, 10 to 75, 15 to 75, 20 to 75, 25 to 75, 30 to 75, 35 to 75, 40 to 75, 45 to 75, 50 to 75, 55 to 75, 60 to 75, 65 to 75, 70 to 75, 5 to 100, 10 to 100, 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, 95 to 100, or more nucleotides or residues in length. In some cases, the guide RNA is from 10 to 15, 10 to 20, 10 to 30, 10 to 40, or 10 to 50 residues in length.

### Definitions

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

“Recombinant” as used herein to describe a nucleic acid molecule means a polynucleotide of retron, genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the polynucleotide of interest is cloned and then expressed in transformed organisms, for example, as described herein. The host organism expresses the foreign nucleic acids to produce the RNA, RT-DNA, or protein under expression conditions.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide or a selected RNA, is a nucleic acid molecule which is transcribed (in the case of DNA) in RNA and/or translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic ncRNA, mRNA, genomic DNA sequences from retron, viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 20 nucleotides, typically 20 nucleotides or more, 40 nucleotides or more, 60 nucleotides or more, 80 nucleotides or more, or 100 nucleotides or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or RNA or cause other adverse consequences. That is, a nucleic acid or peptide of this invention is purified if it is substantially free of cellular material, viral material, or culture medium when obtained from nature or when produced by recombinant DNA techniques, or free from chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Substantially purified” generally refers to isolation of a substance (nucleic acid, compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign nucleic acids by a cell. A cell has been “transfected” when exogenous nucleic acids have been introduced inside the cell membrane. A number of transfection techniques are generally available. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous nucleic acids moieties into suitable host cells. The term refers to both stable and transient uptake of the genetic material and includes uptake of peptide-linked or antibody-linked DNAs. However, the methods described herein involve transfection of RNA into cells.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Expression” refers to detectable production of a gene product by a cell. The gene product may be a transcription product (i.e., RNA or ncRNA), which may be referred to as “gene expression”, or the gene product may be a translation product of the transcription product (i.e., a protein), depending on the context.

“Mammalian cell” refers to any cell derived from a mammalian subject suitable for transfection with retron nucleic acids or vector systems comprising retron nucleic acids, as described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

The term “derived from” is used herein to identify the original source of a molecule but is not meant to limit the method by which the molecule is made which can be, for example, by chemical synthesis or recombinant means. A polynucleotide or nucleic acid “derived from” a designated sequence refers to a polynucleotide or nucleic acid that includes a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The terms “hybridize” and “hybridization” refer to the formation of complexes between nucleotide sequences which are sufficiently complementary to form complexes via Watson-Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

The term “donor polynucleotide” or “donor DNA” refers to a nucleic acid or polynucleotide that provides a nucleotide sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide (donor DNA). The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. For example, the nucleotide sequence comprising the intended edit can be integrated into the genomic DNA by HDR or recombineering at the genomic target locus recognized (i.e., sufficiently complementary for hybridization) by the 5′ and 3′ homology arms.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In one embodiment, the protospacer is a defined synthetic DNA. In some embodiments, the defined synthetic DNA is at least 3, 5, 10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than forty prokaryotes (see e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

Before the present disclosure is further described, it is to be understood that the disclosed subject matter is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

The following Examples illustrate some of the materials, methods, and experiments that were used or performed in the development of the invention.

### Example 1: Materials and Methods

This example illustrates some of the materials and methods used in the development of the invention.

A segment of a plasmid encoding an Eco2 retron ncRNA was amplified to extract the template for transcription of the ncRNA. In particular, to produce an expression cassette that encoded the ncRNA, the segment encoding retron ncRNA within the plasmid was amplified by PCR including a T7 promoter site operably linked the to the ncRNA coding region (appended on the 5′ side of retron ncRNA coding region). In vitro transcription was performed using this amplified ncRNA template DNA.

The in vitro transcribed ncRNA was then transfected into cells of the stable HEK293T cell line that included a retron (Eco2) reverse transcriptase expression cassette. First, Eco2 reverse transcriptase expression was induced in the HEK293T cells by 1 ug/mL doxycycline for 24 hours at 37° C. in 24 well plates prior to transfection. Half the plate is not induced to serve as a negative control. Second, the HEK293T cells were transfected using two different methods:
(a) using Lipofectamine™ 3000 with 5×10⁻⁵ nmol oligo spike-in and between 0 to 0.1 nmol Eco2 ncRNA/well or
(b) using the Neon Transfection System with 1×10⁻³ nmol oligo spike-in and between 0 to 0.02 nmol Eco2 ncRNA/well.
   Synthesized DNA was spiked-in during both methods, to facilitate interpretation of the raw qPCR data.

After 24 hours, the cells were harvested and treated with Proteinase K prior to qPCR analysis to determine the amounts of reverse transcribed DNA (RT-DNA) produced from the ncRNA templates transfected into the cells.

### Example 2: Lipofectamine™ Optimizes Delivery of Retron RNA to Cells

This example illustrates that retron ncRNA is transfected at higher levels into cells using Lipofectamine™ than when ncRNA is transfected by nucleofection. Example 1 describes the methods used for evaluating the transfection procedures.

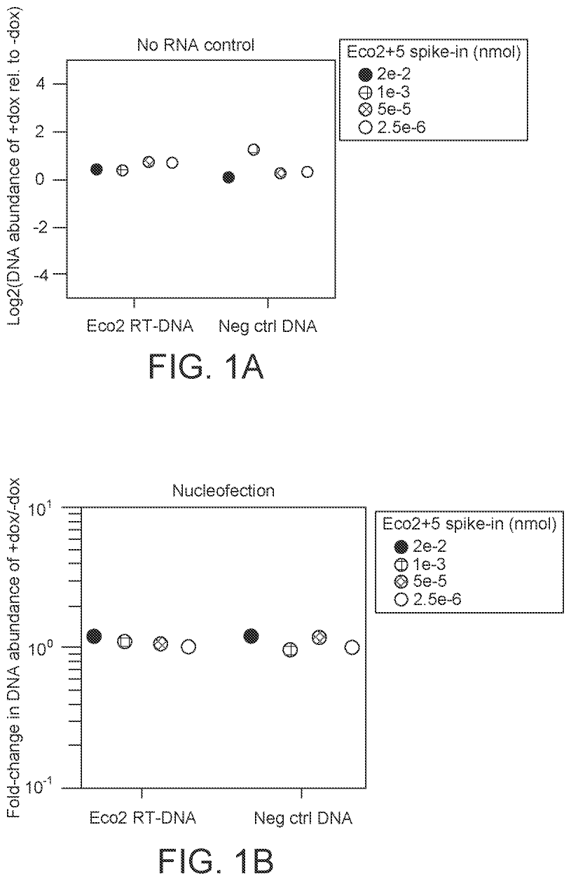

FIG. 1A shows the amounts of reverse transcribed DNA (RT-DNA) made by cells that were transfected with no ncRNA—this is a no RNA control. The cells all expressed an Eco2 reverse transcriptase. The key to the right shows the amounts of synthesized DNA oligos that were spiked into the cells at amounts varying from 2×10⁻² to 2.5×10⁻⁶ nmol. Such synthesized DNA was used to facilitate analysis of the raw qPCR data. As illustrated in FIG. 1A, no detectable ncRNA was reverse transcribed when no ncRNA template is provided.

FIG. 1B shows the amounts of reverse transcribed DNA (RT-DNA) made by cells that were transfected with varying amounts of Eco2 ncRNA by nucleofection. The amounts of Eco2 ncRNA used for nucleofection varied from 0 to 2×10⁻². As illustrated in FIG. 1B, no detectable ncRNA was reverse transcribed when nucleofection was used to import ncRNA into the cells.

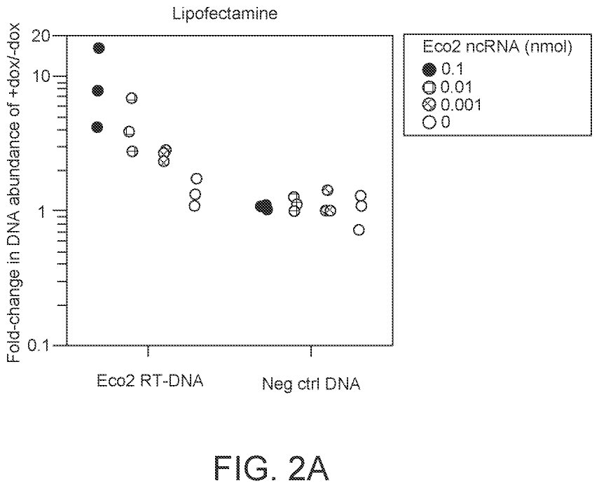

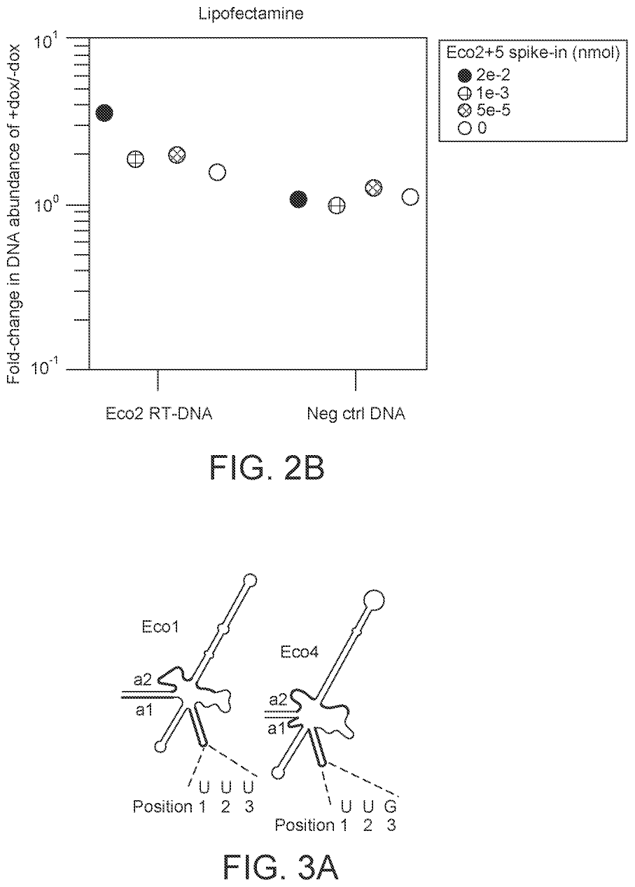

However, as illustrated in FIGS. 2A-2B substantial amounts of reverse transcribed DNA (RT-DNA) were made by cells that were transfected with varying amounts of Eco2 ncRNA by Lipofectamine™. The amounts of RT-DNA were also dose-dependent upon the amounts of ncRNA transfected into the cells. Hence, when 0.1 nmol ncRNA was transfected into cells using Lipofectamine™, significantly more RT-DNA was detected in the cells compared to when 0.001 nmol ncRNA was transfected into cells using Lipofectamine™ (FIG. 2A). Similarly, when 2×10⁻² nmol ncRNA was transfected into cells using Lipofectamine™, significantly more RT-DNA was detected in the cells compared to when 5×10⁻⁵ nmol ncRNA was transfected into cells using Lipofectamine™ (FIG. 2B).

Hence, cationic-lipid transfection reagent based methods efficiently transfect RNA into cells in a dose dependent manner.

### Example 3: ncRNA/RT-DNA Features that can be Modified

The methods described herein can be used to evaluate features of the ncRNA and to that can be modified.

In one experiment, a loop region of the ncRNA was analyzed that was hypothesized to be involved in reverse transcriptase recognition. This loop region had a sequence that was somewhat different in Eco1 and Eco4 retrons (FIG. 3A). As illustrated in FIGS. 3A-3C, this loop was determined to be much more sensitive to modifications in Eco1 than Eco4.

In another experiment, the length of stem regions was evaluated to ascertain optimal stem lengths for retron ncRNAs. As shown in FIGS. 3D-3G, RT-DNA production from both Eco1 and Eco4 ncRNAs was negatively affected by reducing the stem length below about 15 base pairs and by reducing the length of the complementary region at the 5′ and 3′ ends of the ncRNA, termed a1/a2, below about 10 base pairs.

Further experiments have shown that extension of the a1/a2 region can result in more than a ten-fold increase in RT-DNA production, which is the improvement that can be used to increase editing rates, for example, in a variety of cell types, including yeast.

FIG. 4A-4B illustrate some retron features that can be modified. For example, the green segment in FIG. 4A can be a heterologous nucleic acid inserted in a loop of the retron (or ncRNA). Upon reverse transcription of the ncRNA, the RT-DNA can provide a donor DNA (that includes the green segment) with one or more editing nucleotides (star). The stem region of the a1/a2 region can be lengthened as illustrated in FIG. 4B.

### Example 4: Genome Editing Via Exogenous Retron RNA

Editing rates using retron ncRNA/gRNAs provided as exogenous RNA. RNAs were in vitro transcribed and electroporated into HEK293T cells expressing Cas9 and a retron RT of the type matching the RNA (retron-Eco1, retron-Eco3, and retron-Eco5, respectively; FIG. 5). In the Eco1 condition, three bases in the target region were changed (EMX1). In the Eco3 and Eco5 conditions, unique 10 base barcodes were inserted at the target site (EMX1). Precise editing rate was determined by amplifying the genomic site three days after electroporation and Illumina sequencing the resulting amplicons. Negative controls (orange) were an identical experiment performed in a cell line expressing only Cas9 (no retron RT).

Ecol EMX1 editing RNA (donor in bold)
(SEQ ID NO: 10)
GAAATGATAAGATTCCGTATGCGCACCCTTAGCGAGAGGTTTATCATTAA
GGTCAACCTCTGGATGTTGTTTCGGCATCCTGCATTGAATCTGAGTTACT
GTCTGTTTTCCTACAAACGGCAGAAGCTGGAGGAGGAAGGGCCTGAGTCC
GAGCAGAAGAAAAAGTTCTCCCATCACATCAACCGGTGGCGCATTGCCAC
GAAGCAGGCCAATGGGGAGGACATCGATGTCAAGGAAACCCGTTTCTTCT
GACGTAAGGGTGCGCATACGGAATCTTATCAGAGTCCGAGCAGAAGAAGA
AGTTTCAGAGCTATGCTGGAAACAGCATAGCAAGTTGAAATAAGGCTAGT
CCGTTATCAACTTGAAAAAGTGGCACCGAGTCGGTGCTTTTTTTTTTTT
Eco3 EMX1 editing RNA (donor in bold)
(SEQ ID NO: 11)
GAAATGATAAGATTCCGTAAGAGCCAAACCTAGCATTTTATGGGTTAATA
GCCCATCGGGCCATGAGTCATGGTTTCGCCTAGTATTTTAGCTATGCCCG
TCGTTCAGTTCGCTGAACAAACGGCAGAAGCTGGAGGAGGAAGGGCCTGA
GTCCGAGCAGAAGAATTACGTCTGCAAAGTTCTCCCATCACATCAACCGG
TGGCGCATTGCCACGAAGCAGGCCAATGGGGAGGACATCGATGTCATCAG
CGAACTGATCGACGTGCTCAAGTAGGTTTGGCTCTTACGGAATCTTATCA
GAGTCCGAGCAGAAGAAGAAGTTTCAGAGCTATGCTGGAAACAGCATAGC
AAGTTGAAATAAGGCTAGTCCGTTATCAACTTGAAAAAGTGGCACCGAGT
CGGTGCTTTTTTTTTTTT
Eco5 EMX1 editing RNA (donor in bold)
(SEQ ID NO: 12)
GAAATGATAAGATTCCGTACGCCAGCAGTGGCAATAGCGTTTCCGGCCTT
TTGTGCCGGGAGGGTCGGCGAGTCGCTGACTTAACGCCAGTAGTATGTCC
ATATACCCAAAGTCGCTTCATTGTAACAAACGGCAGAAGCTGGAGGAGGA
AGGGCCTGAGTCCGAGCAGAAGAATCCTTCGAGCAAAGTTCTCCCATCAC
ATCAACCGGTGGCGCATTGCCACGAAGCAGGCCAATGGGGAGGACATCGA
TGTCATACAGTTACGCGCCTTCGGGATGGTTTAATGGTATTGCCGCTGTT
GGCGTACGGAATCTTATCAGAGTCCGAGCAGAAGAAGAAGTTTCAGAGCT
ATGCTGGAAACAGCATAGCAAGTTGAAATAAGGCTAGTCCGTTATCAACT
TGAAAAAGTGGCACCGAGTCGGTGCTTTTTTTTTTTT

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method to produce one or more cells containing reverse transcribed DNA (RT-DNA) copies of at least one retron non-coding RNA (ncRNA) comprising contacting one or more cells with an RNA transcript coding for at least one retron non-coding RNA (ncRNA), wherein the one or more cells express a reverse transcriptase (RT).

2. The method of claim 1, wherein the one or more cells stably or transiently expresses RT.

3. The method of claim 1, wherein the RT is expressed from an expression plasmid introduced into the one or more cells.

4. The method of claim 1, wherein the RT is expressed from an RNA transcript coding for RT, wherein the RNA transcript coding for RT is synthesized in vitro prior to introducing said RNA transcript into the one or more cells.

5. The method of claim 1, further comprising contacting the cells with an RNA transcript coding for an mRNA-guided nuclease and/or an RNA transcript coding for guide RNA.

6. (canceled)

7. (canceled)

8. (canceled)

9. (canceled)

10. The method of claim 1, wherein the retron ncRNA is a modified retron ncRNA compared to a wild type retron non-coding RNA.

11. The method of claim 10, wherein the modified retron ncRNA comprises a sequence for a donor DNA.

12. The method of claim 1 wherein the RT is a retron RT.

13. A method of genetically modifying a cell comprising:
introducing an RNA transcript coding for a modified retron non-coding RNA (ncRNA), an mRNA transcript coding for a retron reverse transcriptase, an RNA transcript coding for an mRNA-guided nuclease and an RNA transcript coding for guide RNA into the cell,
wherein the modified retron ncRNA transcript comprises a sequence for a donor DNA;
wherein the RNA transcript(s) are synthesized in vitro prior to introducing said RNA transcript(s) into cells,
wherein the RNA-guided nuclease forms a complex with the guide RNA, wherein said guide RNA directs the complex to a genomic target locus, wherein the RNA-guided nuclease creates a double-stranded break in genomic DNA of the cell at the genomic target locus, and the donor sequence becomes integrated at the genomic target locus of the cell.

14. The method of claim 13, wherein the sequence for the donor DNA comprise one or more variant nucleotides compared to the genomic DNA target locus sequence.

15. (canceled)

16. (canceled)

17. (canceled)

18. (canceled)

19. (canceled)

20. The method of claim 13, wherein the RT is a retron RT.

21. The method of claim 5, wherein the mRNA-guided nuclease is Cas nuclease.

22. (canceled)

23. The method of claim 10, wherein the modified ncRNA comprises an elongated stem region.

24. The method of claim 10, wherein the modified ncRNA comprises an elongated loop region.

25. The method of claim 10, wherein the modified ncRNA comprises a heterologous RNA.

26. The method of claim 10, wherein the heterologous RNA comprises at least one guide RNA sequence.

27. The method of claim 25, wherein the heterologous RNA comprises at least an RNA sequence for a donor DNA.

28. The method of claim 25, wherein the heterologous RNA comprises at least an RNA sequence, which upon reverse transcription comprises a donor DNA.

29. The method of claim 27, wherein the donor DNA is homologous to a target chromosomal sequence in the cell.

30. The method of claim 1, wherein the cells comprise eukaryotic cells.

31. (canceled)

32. (canceled)

33. (canceled)

34. (canceled)


SEQUENCE LISTING (12 sequences)

SEQ ID NO: 1 - DNA, 169 bp, unidentified
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 2 - DNA, 963 bp, synthetic construct
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, synthetic construct
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - protein, 319 aa, unidentified
KSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 5 - protein, 586 aa, unidentified
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 6 - protein, 312 aa, unidentified
MSIDIETTLQKAYPDFDVLLKSRPATHYKVYKIPKRTIGYRIIAQPTPRVKAIQRDIIEILKQHTHIHDAATAYVDGKNILDNAKIHQSSVYLLKLDLVNFFNKITPELLFKALARQKVDISDTNKNLLKQFCFWNRTKRKNGALVLSVGAPSSPFISNIVMSSFDEEISSFCKENKISYSRYADDLTFSTNERDVLGLAHQKVKTTLIRFFGTRIIINNNKIVYSSKAHNRHVTGVTLTNNNKLSLGRERKRYITSLVFKFKEGKLSNVDINHLRGLIGFAYNIEPAFIERLEKKYGESTIKSIKKYSEGG

SEQ ID NO: 7 - protein, 311 aa, unidentified
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 8 - DNA, 169 bp, synthetic construct; identical to SEQ ID NO: 1
  source 1..169: mol_type = other DNA

SEQ ID NO: 9 - DNA, 199 bp, synthetic construct
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca

SEQ ID NO: 10 - DNA, 399 bp, synthetic construct
  source 1..399: mol_type = other DNA
gaaatgataagattccgtatgcgcacccttagcgagaggtttatcattaaggtcaacctctggatgttgtttcggcatcctgcattgaatctgagttactgtctgttttcctacaaacggcagaagctggaggaggaagggcctgagtccgagcagaagaaaaagttctcccatcacatcaaccggtggcgcattgccacgaagcaggccaatggggaggacatcgatgtcaaggaaacccgtttcttctgacgtaagggtgcgcatacggaatcttatcagagtccgagcagaagaagaagtttcagagctatgctggaaacagcatagcaagttgaaataaggctagtccgttatcaacttgaaaaagtggcaccgagtcggtgctttttttttttt

SEQ ID NO: 11 - DNA, 418 bp, synthetic construct
  source 1..418: mol_type = other DNA
gaaatgataagattccgtaagagccaaacctagcattttatgggttaatagcccatcgggccatgagtcatggtttcgcctagtattttagctatgcccgtcgttcagttcgctgaacaaacggcagaagctggaggaggaagggcctgagtccgagcagaagaattacgtctgcaaagttctcccatcacatcaaccggtggcgcattgccacgaagcaggccaatggggaggacatcgatgtcatcagcgaactgatcgacgtgctcaagtaggtttggctcttacggaatcttatcagagtccgagcagaagaagaagtttcagagctatgctggaaacagcatagcaagttgaaataaggctagtccgttatcaacttgaaaaagtggcaccgagtcggtgctttttttttttt

SEQ ID NO: 12 - DNA, 437 bp, synthetic construct
  source 1..437: mol_type = other DNA
gaaatgataagattccgtacgccagcagtggcaatagcgtttccggccttttgtgccgggagggtcggcgagtcgctgacttaacgccagtagtatgtccatatacccaaagtcgcttcattgtaacaaacggcagaagctggaggaggaagggcctgagtccgagcagaagaatccttcgagcaaagttctcccatcacatcaaccggtggcgcattgccacgaagcaggccaatggggaggacatcgatgtcatacagttacgcgccttcgggatggtttaatggtattgccgctgttggcgtacggaatcttatcagagtccgagcagaagaagaagtttcagagctatgctggaaacagcatagcaagttgaaataaggctagtccgttatcaacttgaaaaagtggcaccgagtcggtgctttttttttttt